(19) United States
(12) Patent Application Publication
(10) Pub. No.: US 2002/0164797 A1
(43) Pub. Date: Nov. 7, 2002

(54) Production of metabolites of interest by co-culture of plant cells and non-plant cells

(76) Inventors: Richard Martin, Rochecorbon (FR); Beatrice Belcour-Castro, La Riche (FR); Pascal Hilaire, Vouvray (FR)

Correspondence Address: Norman H. Stepano, Esquire, BURNS, DOANE, SWECKER & MATHIS, L.L.P., P.O. Box 1404, Alexandria, VA, 22313-1404, US

(21) Appl. No.: 09/985,450

(22) Filed: Nov. 2, 2001

(30) Foreign Application Priority Data
Nov. 3, 2000 (FR) 00/14144

(51) Int. Cl. C12N 5/04; C12N 7/01; C12N 1/18; C12N 1/20

(52) U.S. Cl. 435/410; 435/235.1; 435/252.1; 435/254.1

(57) ABSTRACT

The invention relates to stable in vitro co-cultures of cells of plant origin and phytopathogens, which make it possible to produce plant substances.

The invention also relates to a method for the co-culture of cells of plant origin and phytopathogens in a fermentor to produce substances of interest.

### FIELD OF THE INVENTION

The present invention relates to cell culture methods permitting the synthesis of novel compounds based on the co-culture of plant cells and cells or organisms derived from a different kingdom.

### BACKGROUND AND PRIOR ART

Owing to the richness of their genetic potential, the higher plants and fungi are capable of producing compounds likely to present an interest in many biotechnological applications, in particular in the foodstuffs, agrochemical, pharmaceutical or cosmetic industries. In fact they constitute a potentially inexhaustible source of novel molecules. When such a substance possessing an interesting activity is identified, the question of its synthesis in sufficient quantity to meet existing needs must be considered.

Two principal modes of production are possible: chemical synthesis, which is often quite cheap when the structure is relatively simple, and extraction of the compound from the plant or fungal biomass. Culture in vitro is a technique employed to carry out the synthesis of these substances: being free of climatic hazards and agronomic techniques, it permits uniform production, sometimes in high yields. It also makes possible the activation of certain metabolic pathways repressed during the normal growth of the plant. However, culture in vitro also presents technological challenges such as that of axenic cultures in a fermentor for long periods, with long generation times, for plant cells.

Many molecules of interest produced by plant and fungal cells in culture are derivatives of primary metabolism. These derivatives are not directly necessary for the growth of the organism, possess a great variety of structures and biological activities owing to the diversification of the basic units involved in their synthetic pathways. They are specific and vary from one species to another. Their synthesis does not occur throughout the entire life cycle but is related to a particular phase of the culture: it only starts when this phase is attained, usually during the stationary phase of growth. It is controlled by a set of genes regulating the time and level of expression. The control mechanisms form an integral part of the physiology of the producing organism.

The synthesis of the metabolites under consideration may induce a more or less marked antagonism with the completion of primary metabolism corresponding to cell division. Usually it proceeds in two phases: a step in which biomass is produced, then a step involving the manufacture of the metabolites in a special medium in which the tissues no longer grow.

The culture in vitro of plant cells and/or tissues is an important potential source of secondary metabolites, but their natural synthesis often occurs at a relatively low level: their expression is repressed and it is sometimes necessary to add to the cells specific inducers in order to enable them to express their latent genetic potential and optimise the production.

Extracts of inactivated phytopathogens can thus play the role of elicitors and stimulate the production of interesting molecules. Elicitation consists of inducing an increase in the synthesis of certain secondary metabolites by plant cells; at the time of an infection by an external pathogenic agent, certain genes whose activity is low are stimulated and this induces an increase in secondary metabolism and the synthesis of phytoalexins by the plant. These phytoalexins are antimicrobial substances produced and accumulated in response to the aggression by the phytopathogenic organism. They are compounds derived from secondary metabolism and usually have a low molecular weight (Paxton 1981, Whitehead and Threlfall 1992).

The addition of elicitors to cultures of plant cells is consequently envisaged in order to improve the production of molecules of interest. Two types of elicitors are distinguished:

[symbol] biotic elicitors: inactivated (by autoclaving or freezing) natural extracts such as ground extracts of bacteria, phytopathogenic fungi (carbohydrates derived from their cell wall . . . )

[symbol] abiotic elicitors: constraints and stresses due to the cold, U.V. radiation . . .

Thus the objective of elicitation is to cause the plant cells to produce secondary metabolites of interest in large quantity. In the case in which these metabolites are already expressed naturally, an attempt is made to increase the natural expression by adding extracts of biotic elicitors to the nutrient medium. Conversely, the strains are stressed so that they express their “latent” genetic potential under favourable conditions. Several methods can be considered: the addition of precursors of the metabolites considered, their coupling to elicitors, rendering cells permeable or immobilized.

The table below presents examples of molecules of interest obtained by elicitation and the elicitor used.

TABLE 1
Examples of compounds obtained by elicitation

| | | Compounds | |
|---|---|---|---|
| Plant cells | Elicitor | produced | References |
| Papaver somniferum | Botrytis | Sanguinarine | Part et al. 1992 |
| Papaver somniferum | Colletotrichum | Sanguinarine | Eilert et al. 1984 |
| Papaver bracteatum | Verticillium | Sanguinarine | Aine et Coscia 1988 |
| | dahliae | | |
| Sanguinaria | Verticillium | Sanguinarine | Aine et al. 1993 |
| canadensis | dahliae | | |
| Eschscholtzia | Verticillium | Sanguinarine | Byun et al. 1992 |
| california | dahliae | | |
| Gossypium hirsutum | Verticillium | Phytoalexins | Davis et al. 1992 |
| | dahliae | | |
| Catharanthus roseus | Aspergillus | Alkaloids | G-Hernandez et al. 1991 |
| | niger | | |
| Lycopersicum | Vertiallium | Rishitine | Paxton 1981 |
| esculentum | dahliae | | |
| Carthamus tinctorius | Anabaena | Red pigment | Hanagata et al. 1994 |
| | cylindrica | | |
| Tagetes patula | Aspergillus | Thiophene | Buitelaar et al. 1992 |
| | niger | | Buitelaar et al. 1993 |
| Morinda citrifolia | Pseudomonas | Anthraquinones | Dömenburg et Knorr 1994 a |
| | syringae | Chitinase | Dömenburg et Knorr 1994 b |
| Glycine max | Phytophtora | Jasmonate | Ohta et al. 1997 |
| | megasperma | | |
| Lithospermum | Methyl- | Rosmarinic acid | Mzukarri et al. 1993 |
| erythrorhizon | jasmonade | | |
| Pinus taeda | Ophiostoma | Ethylene | Popp et al. 1997 |
| | minus | | |
| Ruta graveolens | Rhodotorula | Furocoumarins | Bohlmann et al. 1995 |
| | rubra | | |

Thus, under conditions of stress or external aggression, the plants can activate certain metabolic pathways and reveal a genetic potential repressed under conditions of normal growth. For example, it is known that the production of certain quinones is only operative when the plant is aggressed by bacteria or fungi. This is the case for most of the phytopathogens like Verticillium dahliae (fungus) when it parasitises the dahlia and also for the Aspergillus genus. Many bacterial taxons like the genus Erwinia produce the same effects. The attacks of insects (oak gall) or viruses (red pigments on linden leaves) also cause defence reactions capable of generating new coloured molecules. The plant reacts by secreting polyphenol oxidases (laccases and catecholases) which oxidise their polyphenols to quinones. These latter polymerise spontaneously on contact with the oxygen of the air, creating a bacteriostatic and/or fungistatic “cicatricial” film, thus counter-acting the invasiveness of the exogenous aggressor.

In cocoa, there is secretion of phytoalexins (arjunolic acid, cyclo-octa-sulfur, phenols). In cotton, there is induction of HMGR (3-hydro-3-methyl-glutaryl CoA reductase), the first enzyme involved in the primary defence mechanism and the synthesis of terpenes. The plant also produces a sesquiterpenoid (isoprenoid) phytoalexin as resistance factor, and desoxyhemigossypol in response to infection; peroxidation of the lipid membrane, the diminution of the concentration of soluble proteins, variation in the content of the lipids of the roots (fall in the content of total lipids, neutral lipids and phospholipids) and the synthesis of phenolic pigments have also been observed (Yunosova et al., 1989, Li et al., 1995). In the eggplant, an increase in the activities of the β-1,3-glucanase and amylase in the leaves has been observed. In the potato, the response to infection is expressed by hypersensitivity, the synthesis of a phytoalexin (rishidine) and suberisation.

A similar phenomenon is observed with the phytopathogens. When a fungus is found in contact with a plant, its germ tube forms a specialised organ, the appressorium, which serves as base for the penetration of the cuticle of the plant cells. It is the combined action of mechanical pressure and various enzymatic systems which permits this penetration. Thus, Verticillium dahliae produces an extracellular alkaline protease when it grows in liquid medium supplemented by a protein source. It also possesses pecto- and proteolytic enzymes: endo-poly-galacturonase, pectin trans-eliminase, pectin methyl esterase which play a role in the penetration of the host and the survival of the saprophyte but not in the infection and expression of the symptoms of verticillosis. It synthesises ethylene, propyl alcohol, ethyl acetate, methyl acetate (phytotoxin).

The interaction between a plant and a micro-organism thus often leads to a modification of the metabolism of one of the two organisms, even of both organisms at the same time. Moreover, symbiosis may result from the interaction between a plant and certain bacteria or certain fungi. Such natural processes have already been exploited to improve the growth of certain plants. Thus, several examples of interaction between plants and bacteria have been described.

For example, rhizobacteria promote the growth of plants and protect them against pathogenic micro-organisms. Thus, Pseudomonas fluorescens M.3.1 stimulates the growth of maize and limits the harmful effect of Fusarium roseum (Lugtenberg et al. 1991, Benizri et al., 1997).

Another effect of a bacterium promoting the growth of plants has been studied: Pseudomonas sp. strain PsJN makes it possible to increase the resistance of young tomato plants to wilting caused by Verticillium dahliae (Sharma & Novak 1998). Tomato plantlets of a cultivar sensitive to Verticillium dahliae were co-cultured in vitro with the bacterium. They were then infected with V. dahliae and seedlings were colonised in vivo after 3 weeks of growth in the greenhouse. In culture in vitro significant differences were noted between the plants co-cultured with Pseudomonas and the control plants, the degree of protection conferred by bacterial colonisation being a function of the density of the inoculum of V. dahliae. In culture in vivo it is only after 3 weeks of exposure to the pathogen that differences of growth appear. That suggests

TABLE 2
Examples of phytopathogens and botanical families targeted by these latter.

| | | | | Number of |
|---|---|---|---|---|
| | | | | strains of |
| | | | | the genus |
| | Pathogen | | | with the |
| Plants/Family | implicated | Observations | Comments | ATCC |
| Rye/Grasses | Claviceps | Synthesis of ergotamine | Used in | 33 |
| | purpurea | and its derivatives | pharmacy | |
| | fungus | | | |
| Brassicaceae | Leptosphaeria | Synthesis of black | Asexual form of | 45 |
| | maculans | (blackleg) or grey | this fungus: | |
| | fungus | colorants | Phoma lingam | |
| Many botanical | Alternaria sp. | Various colours as a | | 226 |
| families | fungus | function of the species of | | |
| | | fungus and plant | | |
| | | affected | | |
| Many botanical | Fusarium sp. | Various colours as a | | 963 |
| families | fungus | function of the species of | | |
| (Solanacees . . . ) | | fungus and plant | | |
| | | affected | | |
| Essentially | Colletotricum | Anthracnose | | 250 |
| kidney beans | sp. fungus | Colours ranging from red | | |
| and lentils | | to black | | |
| All families | Aspergillus sp. | Aspergiloses | Various colours | 1374 |
| | fungus | (fumagiline . . . ) | obtained by | |
| | | | polyphenols | |
| Essentially | Agrobacterium | Nodules | | 87 |
| leguminous | sp. Bacterium | | | |
| essentially | Erwinia sp. | Yellow to red colours | | 188 |
| Umbelliferae | Bacterium | | | |
| All botanical | Pseudomonas | All colours | | 1120 |
| families | sp. Bacterium | | | |

These phytopathogen/host couples can be used in the co-cultures of the invention.

However, the co-cultures of the present invention are not limited to co-cultures of cells of plant origin in the presence of a natural or supposed phytopathogen of the plant under consideration. For example, a pathogen of a given botanical family can be co-cultured with cells derived from a different botanical family. It is also possible to envisage the co-culture of cells of plant origin with cells or organisms not corresponding to a phytopathogen identified as such.

Indeed, although the only plant cells/phytopathogens interactions characterised so far are related to observable infections in nature, the invention proposes also to carry out co-cultures of plant cells and cells with which they have never been placed in contact, for the purpose of inducing or promoting the synthesis of novel compounds. Under these conditions, synthesis can be the result of de-repression of certain latent metabolic pathways in one or several of the co-cultured types of cell or of any other metabolic process.

In the remainder of this text, the term “phytopathogen” will hence designate any organism or cells other than cells derived from higher plants, for example micro-organisms such as bacteria, archebacteria, cyanobacteria, viruses, protists, yeasts, fungi, whether or not it is an organism capable of infecting a plant naturally, or cells isolated from multicellular organisms, in particular animal cells, provided that the association in vitro established under the conditions of the invention between plant cells and the phytopathogen leads to the production of substances of interest.

The term “authentic phytopathogen” will designate, in the framework of the definition of phytopathogens given above, a phytopathogen identified as such in the literal sense of the prior art, i.e. an organism, in particular a micro-organism or virus known to induce a disease in a plant. In this definition, the term “disease” designates any inhibition of the growth and development, necrosis of tissues or diminution of fertility in a plant. A phytopathogen will be called “authentic” irrespective of the range of its natural host spectrum, provided that this spectrum includes at least one plant species. An authentic phytopathogen can, according to the invention, be co-cultured with plant cells of a species distinct from its natural host(s). Thus, Verticillium dahliae is an authentic phytopathogen because it is known to parasitise naturally a large number of plant species including the dahlia, cotton, potato, cocoa, tomato, eggplant or even the strawberry. In Example 2, this authentic phytopathogen is co-cultured with cells of Ruta graveolens, which is not, however, its preferred natural host.

Where appropriate, the co-cultures of the invention may include plant cells of several types and/or several different phytopathogens. In every case, a co-culture according to the present invention will include at least one cell type of plant origin and a cell type of a different kingdom, this latter being designated by the term “phytopathogen”.

The co-cultures of the present invention are “true” co-cultures, which signifies that the different co-cultured cells and/or organisms are live when they are included in the culture, in contradistinction to elicitation by placing plant cells in contact with inactivated natural extracts.

The co-cultures according to the invention differentiate from cultures in which the live cells are either only plant cells or only phytopathogens, which can be called also “pure cultures”. An example of a pure culture of phytopathogens is the culture of Verticillium dahliae described in Example 2. Another example of a pure culture is a culture of plant cells in the presence of an elicitor such as autoclaved bacteria.

According to a particular embodiment of the invention, the co-culture is stable, which means that the different cell types present are capable of reproducing themselves or at the very least of subsisting in co-culture such that an equilibrium may be established between the different lines (plant and phytopathogen) resulting in the fact that a large number of successive subculturings of the co-culture does not lead to the elimination of one of the lines. In practice, a co-culture can be considered to be stable as soon as all of the cellular types present are alive at the end of a complete growth cycle of the plant cell, allowing the doubling of the reference cell population (when several types of plant cells are present in the co-culture, the growth cycle under consideration will be that of the slowest growing cell). In fact, experience shows that if all of the cell types of a co

The substances of interest produced by the methods of the invention may be designed for several types of industrial applications, in particular in the foodstuffs, agrochemical, pharmaceutical and cosmetic fields. Preferred co-cultures of the invention are those which enable the production of substances of interest for the foodstuffs, agrochemical, pharmaceutical or cosmetic fields.

In a preferred embodiment of a co-culture according to the invention, a substance of interest produced by said co-culture is synthesised more efficiently than in any of the pure cultures in the context of the present invention. The initiation or increase of production of said substance of interest is measured in comparison to the pure culture which is the most efficient for producing said substance. The substance of interest can be released in the culture medium or not. In the latter case, however, a step of cell lysis may be necessary to measure the production of said substance. In a preferred co-culture according to the invention, the increase of synthesis of a substance of interest can be 2- to 3-fold, or 3- to 10-fold. In certain co-cultures, it can be superior to 10-fold, or even superior to 100-fold.

Particular types of substance that may be obtained by the co-cultures of the invention are colouring materials, which can be used in cosmetics. Among these compounds a distinction can be made between dyes and pigments. The dyes are soluble in a solvent and are small molecules which easily penetrate hair. The pigments are insoluble in the medium in which they are used. Their structure is crystalline or amorphous.

The first colouring materials used were of plant (indigo, madder, campeachy wood), animal (cochineal, purple) or mineral (ultramarine) origin.

Table 3 below presents several colouring materials, extracted from plants and fungi

TABLE 3

| Pigments | Chemical family | Natural source | | Colour |
|---|---|---|---|---|
| Luteolin | Flavonoids | Reseda luteola | Resedaceae | Yellow |
| Apigenin | | | | |
| Apioside | Flavonoids | Serratula tinctoria | Compositae | Lemon yellow |
| Kaempferol | | | | |
| Quercetin | Flavonoids | Quercus tinctoria | Fagaceae | Golden yellow |
| Morin | Flavonoids | Morus tinctoria | Moraceae | Golden yellow |
| Myricetol | Flavonoids | Myrica gale | Myriaceae | Intense yellow |
| Quercetin | | | | |
| Rhamnetol | Flavonoids | Rhamnus lycioides | Rhamnaceae | Yellow orange |
| Xanthorhamnosi | | | | |
| de | | | | |
| Berberine | Alkaloids | Berberis vulgaris | Berberaceae | Yellow orange |
| Crocin | Carotenoids | Croccus sativus | Iridaceae | Yellow orange |
| Curcumin | Lignins | Curcuma domestica | Zingiberaceae | Yellow orange |
| Emodol | Anthraquinones | Rumex obtusifolius | Polygonaceae | Yellow orange |
| Chrysophanol | | Rheum rhubarbatum | | |
| Morindone | Anthraquinones | Morinda citrifolia | Rubiaceae | Orange |
| Lawsone | Naphthoquinones | Lawsonia inermis | Lythraceae | Orange |
| Bixin | Carotenoids | Bixa orellana | Bixaceae | Reddish orange |
| Alizarin | Anthraquinones | Rubia tinctorium | Rubiaceae | Red |
| Alkannin | Naphthoquinones | Alkanna tinctoria | Borraginaceae | Red |
| Juglone | Naphthoquinones | Juglans regia | Juglandaceae | Dark red |
| Sanguinarine | Alkaloids | Papaver somniferum | Papaveraceae | Dark red |
| Cyanidol | Anthocyans | Sorghum vulgare | Gramineae | Reddish |
| Pelargonidol | | Papaver rhoeas | Papaveraceae | purple |
| Malvidol | Anthocyans | Vaccinum myrtillus | Ericaceae | Reddish |
| Petunidol | | | | purple |
| Cynodontin | Anthraquinones | Curvularia lunata | Fungus | Blue |
| Flaviolin | Naphthoquinones | Verticillium dahliae | Fungus | Violet |

### Example of Murashige & Skoog Basic Medium (Detailed)

| Skoog macro-elements | 100 ml/l |
|---|---|
| Skoog micro-elements | 1 ml |
| Skoog vitamins | 2 ml |
| Iron EDTA | 10 ml |
| 2,4-D 10⁻⁴ | 10 ml |
| Kinetin 10⁻⁴ | 0.6 ml |
| | (0.06 mg) |
| Sucrose | 30 g |
| Distilled water | qsp 1 litre |
| pH before sterilisation | 5.8 pH |
| Sterilisation 115 or 121° C. for 20 to 40 minutes | |
| To obtain a solid gelosed medium are added: | |
| Agar | 8 g |
| Macro elements in mg/l | SKOOG |
| KNO₃ | 1900 |
| NH₄NO₃ | 1650 |
| MgSO₄, 7H₂O | 370 |
| CaCl₂, 2H₂O | 440 |
| KH₂PO₄ | 170 |
| Micro elements in mg/l | SKOOG |
| CuSO₄, 5H₂O | 0.025 |
| MnSO₄, 1H₂O | 16.9 |
| KI | 0.83 |
| Na₂MoO₄, 2H₂O | 0.25 |
| ZnSO₄, 7H₂O | 10.6 |
| H₃BO₃ | 6.2 |
| CoCl₂, 6H₂O | 0.025 |
| Vitamins mg/l | SKOOG |
| Myoinositol | 100 |
| Nicotinic acid | 0.5 |
| Pyridoxine | 0.5 |
| Thiamine | 0.1 |
| FeSO₄, 7H₂O | 27.8 |
| Na₂ EDTA | 37.3 |

In addition to the question of the co-culture medium, it is necessary to determine whether it is possible to establish stable co-cultures in the sense that an equilibrium exists between the different lines and that none of them is eliminated during the successive subculturings of the co-culture.

Finally, depending on the associations achieved, the production of the desired metabolites may be uncertain and must be verified.

The surprising results obtained and presented in the detailed examples below make it possible to reply to these three questions. These examples show in particular that, contrary to what was expected, it is possible to co-culture plant cells and bacteria stably in a conventional medium for the culture of plant cells and that this co-culture makes possible the production of substances which are not produced efficiently in pure cultures of said plant cells and bacteria (Example 1). Example 2 shows, there again quite contrary to expectation, that the co-culture of plant cells and fungi in a conventional culture medium for plant cells can make possible the synthesis of compounds not present in pure cultures of said plant cells and fungi.

The present invention hence relates to methods for the production of substances of interest by the co-culture of plant cells and live phytopathogens according to the definitions and under the conditions described above for the co-cultures. The methods of the invention can in particular make possible the production of substances of pharmaceutical and/or cosmetic interest, in particular the production of colouring materials.

In a preferred embodiment of the methods of the invention, the co-culture of plant cells and live phytopathogens is stable, and this signifies that an equilibrium has been established between the different lines which remain present and alive during the subculturing of the co-culture. As indicated above, this stability is ensured if all of the cell types of the co-culture are alive at the end of a complete growth cycle of the slowest growing cell type.

In a preferred embodiment of the methods of the invention, the co-culture is carried out in a fermentor or sterile and/or sterilisable closed chamber, stirred and/or shaken. Examples of fermentors which can be used for the methods of the invention are stirrer fermentors of trade mark RUSHTON, AIRLIFT, DRAFT Tube (DRAUGHT Tube in USA) or also of the piston type. In the methods of the invention, the organisms can be grown together or separated by a membrane in a batch, fed batch or continuous system. Physical methods making it possible to separate two cultures by a membrane are described in the patent U.S. Pat. No. 5,665,596.

In a batch system, the cells multiply in the fermentor until the culture medium is exhausted.

| | | |
|---|---|---|
| CuSO₄, 5H₂O | 0.025 | mg/L |
| CoCl₂, 6H₂O | 0.025 | mg/L |
| Na₂MoO₄, 2H₂O | 0.25 | mg/L |
| KI | 0.83 | mg/L |
| ZnSO₄, 7H₂O | 8.6 | mg/L |
| H₃BO₃ | 6.2 | mg/L |
| MnSO₄, 4H₂O | 22.3 | mg/L |
| Myoinositol | 100 | mg/L |
| Nicotinic Acid | 0.5 | mg/L |
| Pyridoxine, HCl (4° C.) | 0.5 | mg/L |
| Thiamine, HCl (4° C.) | 0.1 | mg/L |
| Glycine | 2 | mg/L |
| FeSO₄, 7H₂O | 27.8 | mg/L |
| Sequestrene 330 Fe | 37.3 | mg/L |
| Naphthalene Acetic Acid | 1 | mg/L |
| Kinetin | 0.06 | mg/L |
| Sucrose | 30 | g/L |
| Distilled water | qsp 1 | Litre |
| pH before sterilisation | 5.8 | pH |

Sterilisation depending on volumes: from 15′ to 40′ at 115° C. or 121° C.

The roots are inoculated in a ratio of 5 g of fresh weight for 100 ml of culture.

One of the two cultures has beige roots whereas in the second they are orange-red. The two cultures are stable for several years.

The red culture was filtered and its medium was analysed. This latter contains bacteria. On isolation it was revealed that it was a pure bacterial culture which was identified by the Pasteur Institute as being a Streptococcus sp.

This latter was cultured in the dark at 26.5° C. on LPG medium, which is more favourable for its development and has the following composition:

| | |
|---|---|
| Yeast extract | 5 g/L |
| Glucose | 10 g/L |
| Peptone | 5 g/L |
| Agar | 15 g/L culture on gelose |
| Distilled water | qsp 1 litre |

Sterilisation depending on volumes: from 15′ to 40′ at 115° C. or 121° C. In order to verify that the presence of the red coloration of the Impatiens balsamina strain is actually induced by the bacterium, beige roots (uncontaminated) were infected with the streptococcus cultured on LPG medium. In every case of infection the appearance of the red colour is observed.

The phenomenon is stable on subculturing, and a true and stable co-culture is set up, preserving the red colour in the roots of Impatiens balsamina.

These same bacteria, killed by heat then added to non-pigmented roots produce no change. The table below recapitulates the different assays performed:

TABLE 4

| | Colour of the | |
|---|---|---|
| Cultures | cultures | Observations |
| Uninfected roots (A) | Beige | |
| Streptococcus alone (B) | Beige | |
| A + B | Red | Co-culture stable with time |
| Killed A + B | Beige | Normal growth of B |
| A + killed B | Beige | Normal growth of A |

The phenomenon is hence not due to a classic elicitation but indeed to a true co-culture of live cells.

The first surprising effect observed here is the adaptation of the streptococci to the culture medium of the plant cells.

The second surprising effect is the stability of the co-cultures over time: neither of the two cell lines dominates to the exclusion of the other.

Different determinations of lawsone (yellow/orange) show that the latter can be elicited by killed streptococci but this can not explain the appearance of the vivid red coloration of the roots obtained in co-culture.

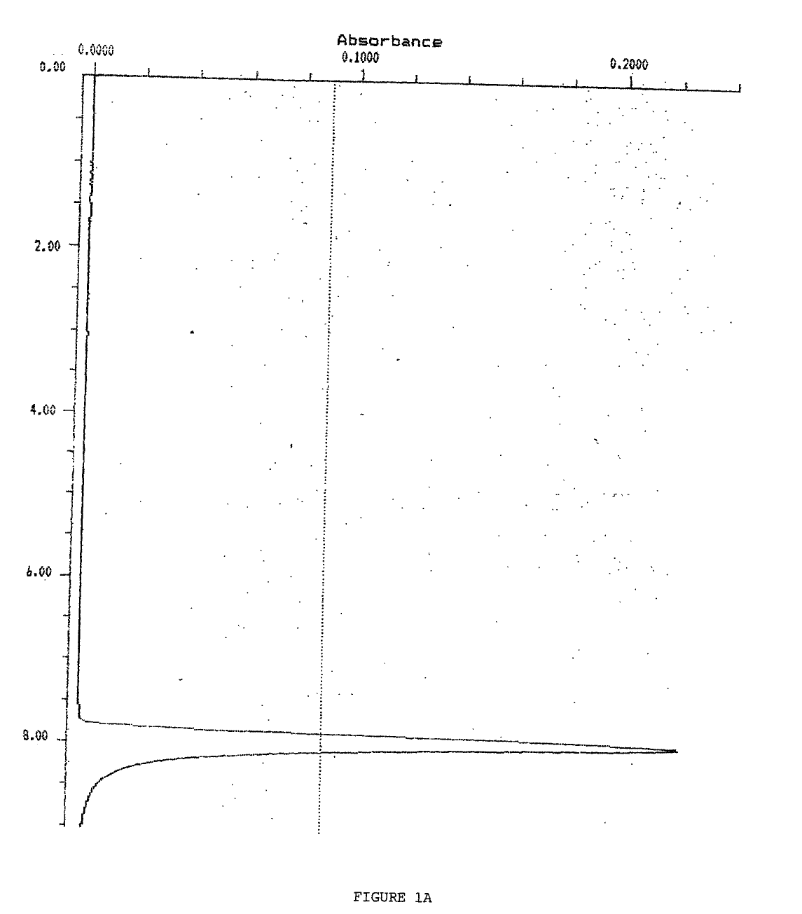

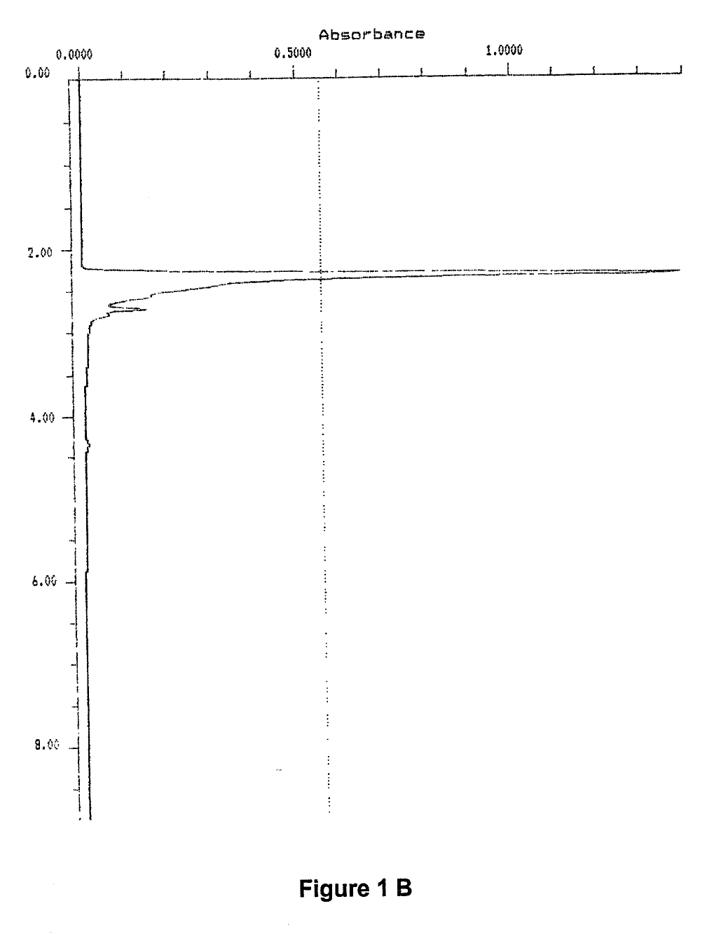

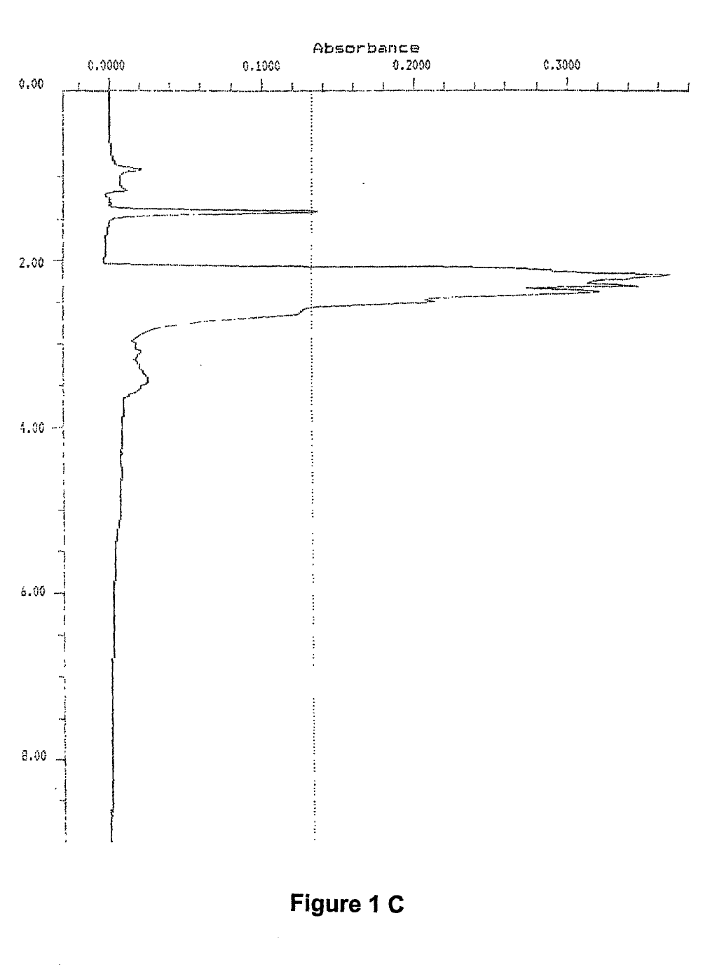

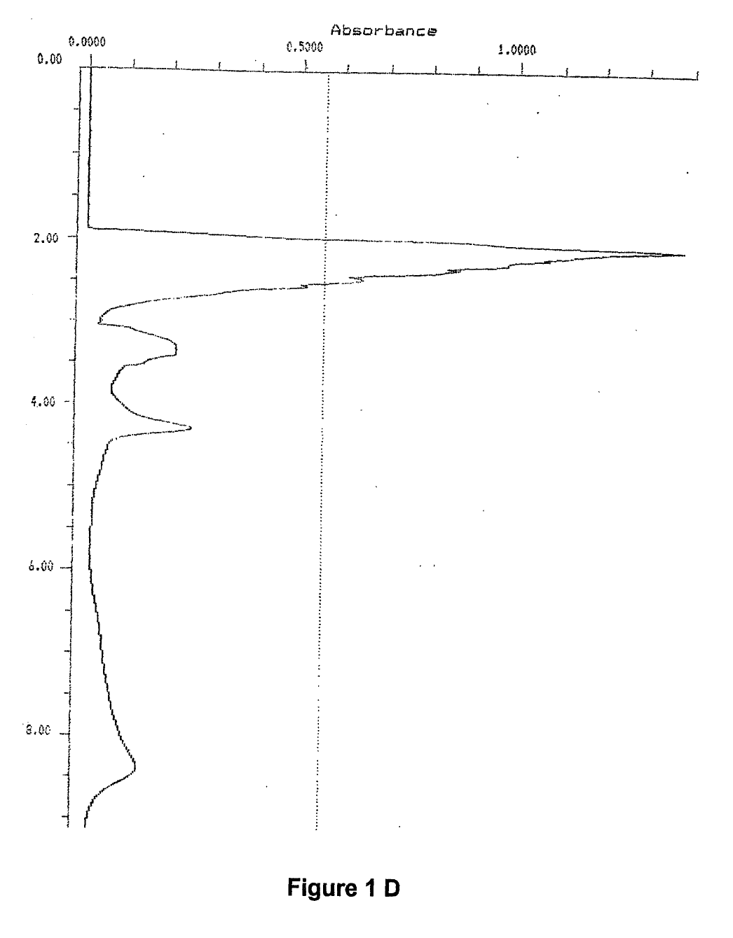

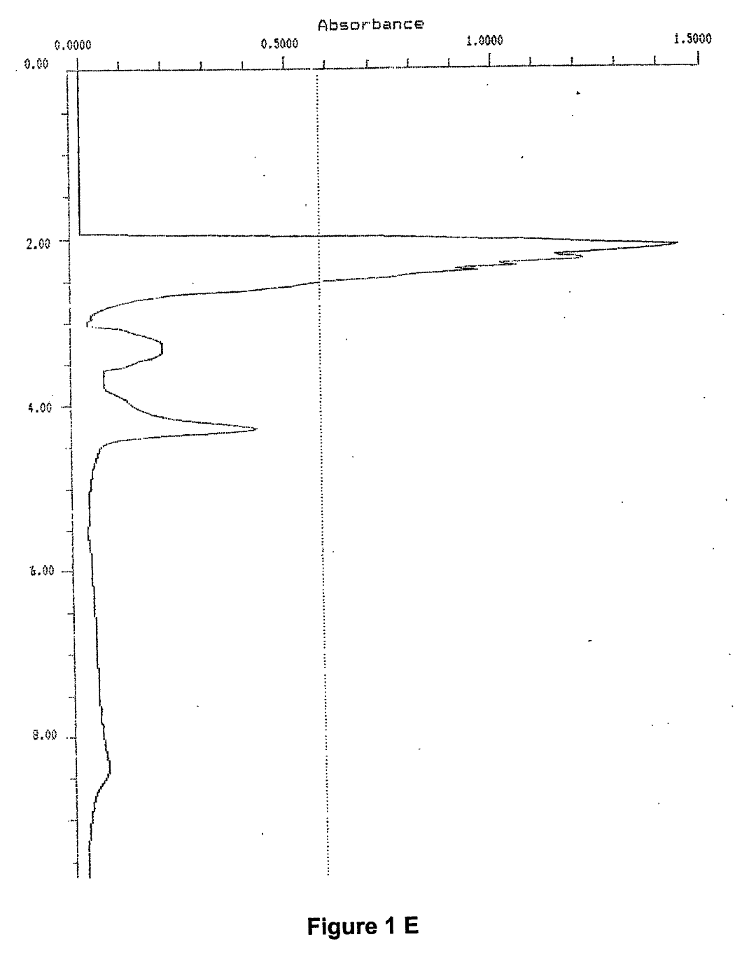

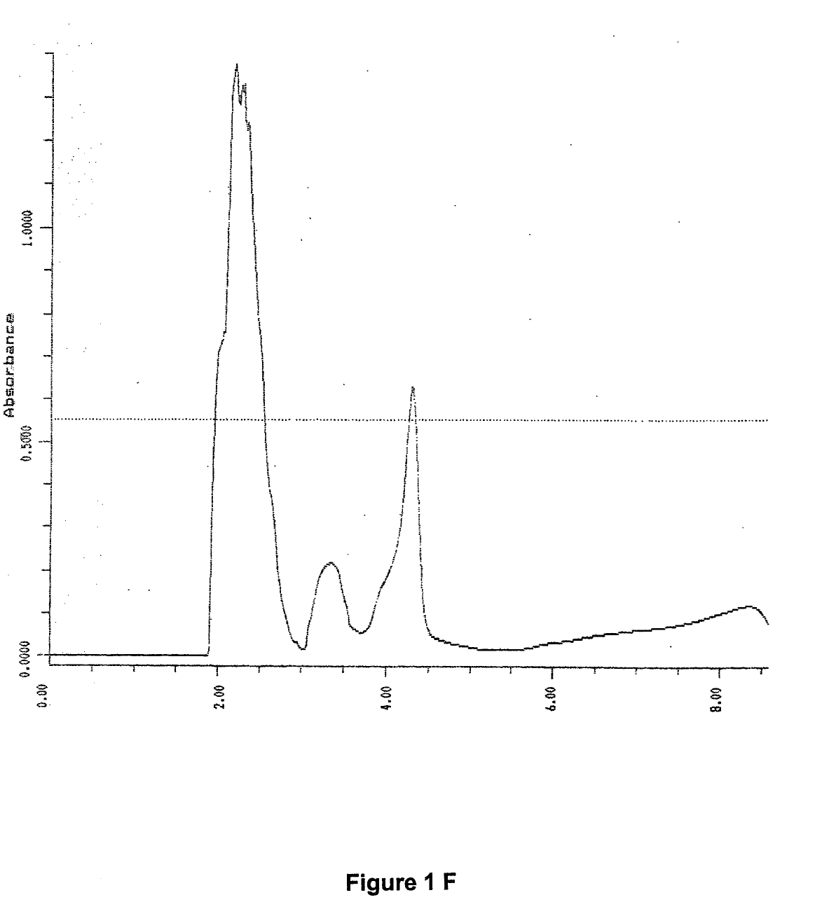

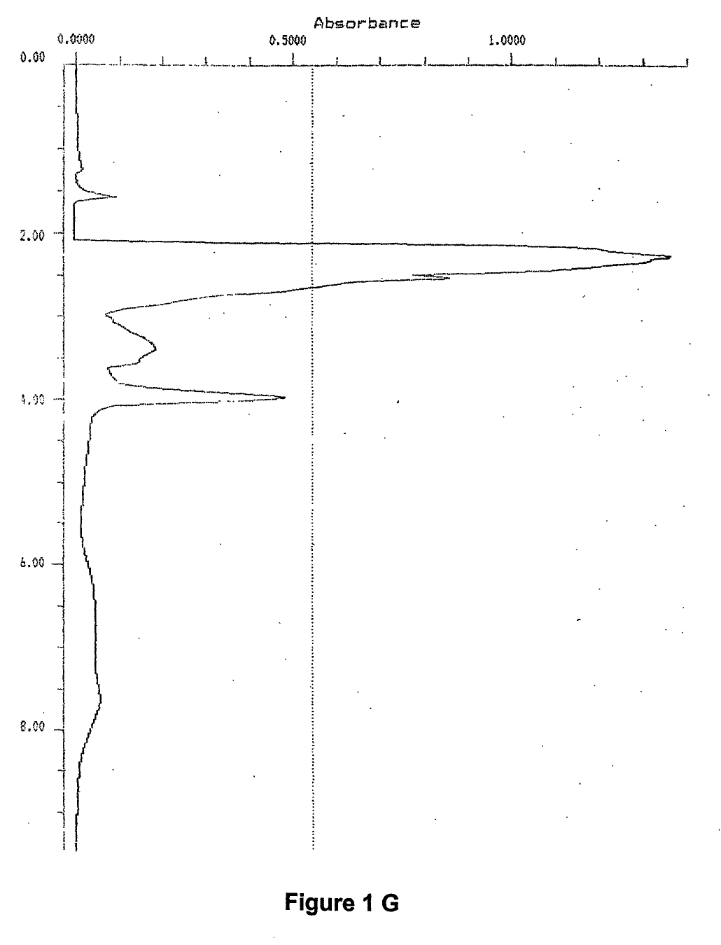

The chromatograms presented in FIG. 1 (ethanolic extraction) show the influence of the different culture conditions on the composition of the roots of Impatiens balsamina.

### EXAMPLE 2

Co-cultures in vitro of Ruta graveolens cells (common rue) and Verticillium dahliae

This example illustrates the possibility of culturing a dedifferentiated cell of plant origin in the presence of a phytopathogenic fungus.

Ruta graveolens is a plant of the family of the Rutaceae which synthesises furocoumarins including psoralen and some of its methoxylated derivatives: 5-MOP (bergaptene), 8-MOP (xanthotoxin) and 5,8-MOP (isopimpinellin). These furocoumarins are secondary metabolites produced in response to an aggression by a phytopathogen. They are thus phytoalexins limiting the proliferation of the phytopathogens.

Verticillium dahliae is a phytopathogenic lower fungus belonging to the family of the Adelomycetes of the order of the Hyphales which parasitises a large number of plant species including dahlia, cotton, potato, cocoa, tomato, eggplant or even the strawberry. Among others it synthesises a naphthoquinone: flaviolin or 2, 5, 7-trihydroxy 1,4-naphthoquinone.

It is grown on PDA medium of composition:

| | |
|---|---|
| Potato mash | 200 g/L |
| Glucose | 20 g/L |
| (Agar | 15 g/L) |
| pH before sterilisation | 5.6 pH |

The cultures are grown in the dark in Petri dishes at 26.5° C. Transfer to liquid medium is carried out in B5 D2, a medium for vegetative growth, in the dark:

Macro-, micro-elements, vitamins and iron of the Gamborg medium

| | |
|---|---|
| 2,4-Dichlorophenoxyacetic acid 10⁻⁴ M | 2 mg/l |
| Sucrose | 30 g/L |
| pH before sterilisation | 5.8 pH |

Ruta graveolens is also cultured on B5 D2 medium in the light.

Pure cultures of each of the cellular entities are prepared as are cultures elicited by heat-killed cells of the other type and finally the true co-culture between Ruta graveolens and Verticillium dahliae is carried out.

Each cellular entity is extracted, then analysed. The results are the following:

TABLE 5

| | | Production of |
|---|---|---|
| | Production of flaviolin | furocoumarins in |
| Type of culture | in Verticillium dahliae | Ruta graveolens |
| Verticillium dahliae brm 1 | 6 mg/L (16 d of culture) | — |
| alone | 6 mg/L (16 d of culture) | |
| PDB medium | | |
| B5D2 medium | | |
| Verticillium dahliae brm | 13,5 mg/L | — |
| 1 + Autoclaved cells of | | |
| Ruta graveolens | | |
| Verticillium dahliae brm | 6 mg/L (16 d of culture) | — |
| 1 + Frozed cells of Ruta | | |
| graveolens (without co- | | |
| culture) | | |
| Verticillium dahliae brm 1 | — | 4 μg/g DM |
| autoclaved + Ruta | | (10 d of culture) |
| graveolens | | |
| Verticillium dahliae brm | 15 mg/L | 40 μg/g DM |
| 1 + Ruta graveolens B5D2 | | (10 d of culture) |
| medium true co-culture | | |
| Ruta graveolens alone | — | 13 μg/g DM |
| B5D2 medium | | (10 d of culture) |

Flaviolin: extracellular

Furocoumarins: intracellular

DM: dry matter in the plant cell

The assays were performed in illuminated culture, more adapted to the growth of the R. graveolens strain.

These experiments show that the production of flaviolin is optimised under conditions of true co-culture. Furthermore, the frozen cells (non-viable) give the same results as the pure fungal culture. This result clearly shows that the live/live interaction is necessary to increase the synthesis by a factor of 2.5. Autoclaving leads to a denaturation of the constituents of the plant cell. These latter behave as less efficient elicitors than true co-culture.

As regards the production of furocoumarins, these results show that the addition of killed fungus to the Ruta graveolens culture has a reverse effect to that desired, because only a third of the furocoumarins are produced that are obtained in pure culture. The co-culture makes it possible to produce three times more furocoumarins than the pure culture.

The results thus clearly show the effects of the live/live interaction which makes possible an increase of the biosynthesis of compounds in the two cell lines. These compounds are of very different kinds: colouring materials and phytoalexins, thus showing the great potential of this type of technique.

1. Stable in vitro co-culture of cells of plant origin and phytopathogens, wherein said co-culture produces substances of interest.

2. The co-culture according to claim 1, wherein the cells of plant origin are individually separated or organised in multicellular structures.

3. The co-culture according to claim 1, wherein the plant cells are dedifferentiated.

4. The co-culture according to claim 1, wherein the phytopathogens are archebacteria, bacteria, protists, fungi, animal cells, insects, viruses or yeasts.

5. The co-culture according to claim 1, in which the phytopathogens are prokaryotic cells.

6. The co-culture according to claim 1, in which the phytopathogens are fungi.

7. The co-culture according to claim 1, in which the phytopathogens are viruses.

8. The co-culture according to claim 1, in which the phytopathogens are authentic phytopathogens.

9. The co-culture according to claim 1, in which the phytopathogens are yeasts.

10. The co-culture according to claim 1, in which the plant cells are Impatiens balsamina roots and the phytopathogen is Streptococcus sp.

11. The co-culture according to claim 1, in which the plant cells are dedifferentiated Ruta graveolens cells and the phytopathogen is Verticillium dahliae.

12. The co-culture according to claim 1, wherein the culture medium is initially completely defined.

13. The co-culture according to claim 1, which produces a substance of interest for the foodstuffs, agrochemical, pharmaceutical or cosmetic field.

14. The co-culture according to claim 1, wherein a substance of interest produced by said co-culture is synthesised more efficiently than in any of the pure cultures of either only plant cells or only phytopathogens of said co-culture, the increase of production of said substance of interest being from 2- to 3-fold, or from 3- to 10-fold, or superior to 10-fold, or even superior to 100-fold, compared to the pure culture which is the most efficient for producing said substance.

15. Method for the production in a fermentor of substances of interest by co-culture of plant cells and live phytopathogens.

16. The method of claim 15, for the production of substance of interest for the foodstuffs, agrochemical, pharmaceutical or cosmetic field.

17. The method of claim 15, for the production of a colouring material.

18. The method of claim 15, in which the co-culture is stable.

19. The method of claim 15, in which the co-culture is carried out in a fermentor or closed chamber, sterile and/or sterilisable, stirred and/or shaken.

20. The method of claim 15, in which the organisms are grown together or separated by a membrane in a batch, fed batch or continuous system.

21. The method of claim 15, in which the culture medium is initially completely defined.

22. The method of claim 15, in which the plant cells are isolated or organised in multicellular structures.

23. The method of claim 15, in which the plant cells are dedifferentiated.

24. The method of claim 15, in which the phytopathogens are selected from archebacteria, bacteria, protists, fungi, animal cells, insects or viruses.

25. The method of claim 15, in which the phytopathogens are authentic phytopathogens.

26. The method of claim 15, comprising the following steps:
A. Inoculation of a culture medium with plant cells and selected phytopathogens,
B. Co-culture of the plant cells and the phytopathogens for 1 to 30 days in batch, and in continuous mode throughout the production,
C. Recovery of the substance of interest from the culture medium.

27. The method according to claim 15, for the production of a colouring material by co-culture of Impatiens balsamina and Streptococcus sp.

28. The method according to claim 15, in which the co-culture of plant cells and phytopathogens produces a substance of interest more efficiently than any of the pure cultures of either only plant cells or only phytopathogens of said co-culture, wherein the increase of production of said substance of interest in said co-culture is from 2- to 3-fold, or from 3- to 10-fold, or superior to 10-fold, or even superior to 100-fold, compared to the production of said substance in the pure culture which is the most efficient for producing said substance.

29. The method according to claim 15, in which the co-culture of plant cells and phytopathogens produces substances which are not efficiently produced in a pure culture.

30. The method according to claim 28, wherein said substances form part of the group comprising the phytoalexins, the quinones and their derivatives, lawsone, the polyphenol oxidases, the furocoumarins, the phytochelatins, the peptides or the proteins.

31. Phytoalexins and colouring materials obtained by the method of claim 30.
